SEQUENCE LISTING (13 sequences)

SEQ ID NO: 1 - DNA, 25 bp, artificial sequence
  primer
acagctatcgcgattgcagtggcac

SEQ ID NO: 2 - DNA, 76 bp, artificial sequence
  primer
ggaagatctagaggaaccacccccaccaccgcccgagccaccgccaccagaggaagttcgtttgagttccaccttg

SEQ ID NO: 3 - DNA, 39 bp, artificial sequence
  primer
ggtggttcctctagatcttccgaggtgcagctgctcgag

SEQ ID NO: 4 - DNA, 84 bp, artificial sequence
  primer
cctccagacgggcgatgcggccaccgcaggaagagcccggtggggtagacggtttcggactagtggagacggtgacggtggtcc

SEQ ID NO: 5 - DNA, 87 bp, artificial sequence
  primer
actgccaacatgctgcgcgaacaggtggcacagctgaaacagaaagttatgaaccatggcggttgtgctagtggccaggccggccag

SEQ ID NO: 6 - DNA, 23 bp, artificial sequence
  primer
gcttacaatttcccagatctgcg

SEQ ID NO: 7 - DNA, 84 bp, artificial sequence
  primer
tcgcgcagcatgttggcagtggacgccagctcggagttctgagctttcagggttttcactttttcctccagacgggcgatgcgg

SEQ ID NO: 8 - DNA, 5149 bp, artificial sequence
  vector
gggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagctgtacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaattgaattcaggaggaatttaaaatgaaaaagacagctatcgcgattgcagtggcactggctggtttcgctaccgtggcccaggcggccgagctcggccatggctggttgggcagcgagtaataacaatccagcggctgccgtaggcaataggtatttcattatgactgtctccttggcgactagctagtttagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgttactaatgatggtgatggtgatggctagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcgccggttcggggaagtagtccttgaccaggcagcccagggccgctgtgcccccagaggtgctcttggaggagggtgccagggggaagaccgatgggcccttggtggaggctgcggagacggtgaccgtggtaccagcagaaacctggccaggctcccaggctcctcatctatggtacatccagcagggccactggcatcccagacaggttcagtggcagtgggtctgggacagacttcactctcaccatcagcagactggagcctgaagattttgcagtgtactactgtcagcagtatggtggctcaccgtggttcggccaagggaccaaggtggaactcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacggaggagagtgttaattctagataattaattaggaggaatttaaaatgaaatacctattgcctacggcagccgctggattgttattactcgctgcccaaccagccatggccgaggtgcagctgctcgagatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcacttgtctaaaggtcagttgaaagagttcctcgacgctaacctggcgtacccgtacgacgttccggactacggttctactagtccgaaaccgtctaccccaccgggctcttcctgcggtggccgcatcgcccgtctggaggaaaaagtgaaaaccctgaaagctcagaactccgagctggcgtccactgccaacatgctgcgcgaacaggtggcacagctgaaacagaaagttatgaaccatggcggttgtgctagtggccaggccggccagcaccatcaccatcaccatggcgcatacccgtacgacgttccggactacgcttcttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaattaatttaagcggccgcagatct

SEQ ID NO: 9 - DNA, 270 bp, artificial sequence
  portion of vector
actagtccgaaaccgtctaccccaccgggctcttcctgcggtggccgcatcgcccgtctggaggaaaaagtgaaaaccctgaaagctcagaactccgagctggcgtccactgccaacatgctgcgcgaacaggtggcacagctgaaacagaaagttatgaaccatggcggttgtgctagtggccaggccggccagcaccatcaccatcaccatggcgcatacccgtacgacgttccggactacgcttcttaggagggtggtggctctgag

SEQ ID NO: 10 - protein, 81 aa, artificial sequence
  amino acid sequence encoded by sequence ID 9
PKPSTPPGSSCGGRIARLEEKVKTLKAQNSELASTANMLREQVAQLKQKVMNHGGCASGQAGQHHHHHHGAYPYDVPDYAS

SEQ ID NO: 11 - DNA, 1190 bp, artificial sequence
  light chain stuffer
gagctcggccatggctggttgggcagcgagtaataacaatccagcggctgccgtaggcaataggtatttcattatgactgtctccttggcgactagctagtttagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgttactaatgatggtgatggtgatggctagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcgccggttcggggaagtagtccttgaccaggcagcccagggccgctgtgcccccagaggtgctcttggaggagggtgccagggggaagaccgatgggcccttggtggaggctgcggagacggtgaccgtggtaccagcagaaacctggccaggctcccaggctcctcatctatggtacatccagcagggccactggcatcccagacaggttcagtggcagtgggtctgggacagacttcactctcaccatcagcagactggagcctgaagattttgcagtgtactactgtcagcagtatggtggctcaccgtggttcggccaagggaccaaggtggaactcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacggaggagagtgttaattctaga

SEQ ID NO: 12 - DNA, 370 bp, artificial sequence
  heavy chain stuffer
ctcgagatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcacttgtctaaaggtcagttgaaagagttcctcgacgctaacctggcgtacccgtacgacgttccggactacggttctactagt

SEQ ID NO: 13 - DNA, 195 bp, artificial sequence
  Jun dimerization cassette
actagtccgaaaccgtctaccccaccgggctcttcctgcggtggccgcatcgcccgtctggaggaaaaagtgaaaaccctgaaagctcagaactccgagctggcgtccactgccaacatgctgcgcgaacaggtggcacagctgaaacagaaagttatgaaccatggcggttgtgctagtggccaggccggccag

(19) United States
(12) Patent Application Publication
(10) Pub. No.: US 2005/0191749 A1
(43) Pub. Date: Sep. 1, 2005

(54) Novel plasmid vectors

(76) Inventors: Katherine S. Bowdish, Del Mar, CA (US); Shana Fredrickson, Solana Beach, CA (US)

Correspondence Address: Mark Farber;Alexion Pharmaceuticals Inc, 352 Knotter Drive, Cheshire, CT, 06410, US

(21) Appl. No.: 10/433,281

(22) PCT Filed: Dec. 7, 2001
(86) PCT No.: PCT/US01/46514

§ 371 Date: Jun. 2, 2003

Related U.S. Application Data
(60) Provisional application No. 60/254,410, filed on Dec. 8, 2000.

(51) Int. Cl. C12N 15/74

(52) U.S. Cl. 435/476

(57) ABSTRACT

Phagemid vectors incorporating dimerization domains are provided which allow efficient production of biologically active polypeptides that may require dimerization for their biological activity.

### RELATED APPLICATIONS

This application claims priority to U.S. Provisional Application No. 60/254,410 filed Dec. 8, 2000, the disclosure of which is incorporated herein by this reference.

### BACKGROUND

1. Field of the Invention

This invention relates to cloning vectors. More specifically the invention relates to plasmids useful in the cloning and expression of foreign genetic information.

2. Background of the Invention

Plasmids are extrachromosomal genetic elements and are typically capable of autonomous replication within their hosts. Bacterial plasmids range in size from 1 Kb to 200 Kb or more and encode a variety of useful properties. Plasmid encoded traits include resistance to antibiotics, production of antibiotics, degradation of complex organic molecules, production of bacteriocins, such as colicins, production of enterotoxins, and production of DNA restriction and modification enzymes. Although plasmids have been studied for a number of years in their own right, particularly in terms of their replication, transmissibility, structure and evolution, with the advent of genetic engineering technology the focus of plasmid research has turned to the use of plasmids as vectors for the cloning and expression of foreign genetic information. In its application as a vector, the plasmid should possess one or more of the following properties. The plasmid DNA should be relatively small but capable of having relatively large amounts of foreign DNA incorporated into it. The size of the DNA insert is of concern in vectors based on bacteriophages where packing the nucleic acid into the phage particles can determine an upper limit. The plasmid should be under relaxed replication control. That is, where the replication of the plasmid molecule is not strictly coupled to the replication of the host DNA (stringent control), thereby resulting in multiple copies of plasmid DNA per host cell. The plasmid should express one or more selectable markers, such as the drug resistance markers, mentioned above, to permit the identification of host cells which contain the plasmid and also to provide a positive selection pressure for the maintenance of the plasmid in the host cell. Finally the plasmid should contain a single restriction site for one or more endonucleases in a region of plasmid which is not essential for plasmid replication. It is particularly useful if such a site is located within one of the drug resistance genes thereby permitting the monitoring of successful integration of the foreign DNA segment by insertional inactivation. For example, when a plasmid contains two drug resistance genes and one of the genes contains a single restriction endonuclease site, the foreign DNA when ligated into that site will interrupt the expression of the drug resistance gene, thus converting the phenotype of the host from double drug resistance to single drug resistance. A vector as described above is useful, for example, for cloning genetic information, by which is meant integrating a segment of foreign DNA into the vector and reproducing identical copies of that information by virtue of the replication of the plasmid DNA.

The next step in the evolution of vector technology was the construction of so-called expression vectors. These vectors are characterized by their ability not only to replicate the inserted foreign genetic information but also to promote the transcription of the genetic information into mRNA and its subsequent translation into protein. This expression requires a variety of regulatory genetic sequences including but not necessarily limited to promoters, operators, transcription terminators, ribosomal binding sites and protein synthesis initiation and termination codons. These expression elements can be provided with the foreign DNA segment as parts thereof or can be integrated within the vector in a region adjacent to a restriction site so that when a foreign DNA segment is introduced into the vector it falls under the control of those elements to which it is now chemically joined.

Hybrid vectors have been constructed which permit the cloning and/or expression of foreign genetic information in more than one host. These biphasic or shuttle vectors are characterized as having separate origins of replication (replicons) to permit replication of the plasmid in the desired host; further, in the case of expression vectors, it may be required to have two sets of regulatory elements, each specific for the intended host. Such duplication of regulatory elements is not always required as it may be possible for a single promoter to be able to function in both of the desired hosts. Regardless of the type of biphasic vector, be it either a cloning or expression vector, it may be advantageous to have at least two selectable markers, one permitting selection in each of the contemplated hosts.

Vectors known as phagemids have been produced which are utilized, e.g., in connection with large combinatorial libraries of antibodies having related or diverse immunospecificities. One series of well-known phagemid vectors is the pComb family of phagemids. For example, a well-known pComb vector is pComb3X (GenBank accession No. AF268281). Phagemid pRL4 is similar to pComb3X with altered stuffer regions. A plasmid map illustrating pRL4 is provided in FIG. 1. These vectors may be used to display expression products on the surface of packaged phage particles. pRL4 is a modified version of pComb3H (Barbas and Burton (1994) Monoclonal Antibodies from Combinatorial Libraries. Cold Spring Harbor Laboratory Course Manual, Cold Spring Harbor, N.Y.; Burton and Barbas, Advances in Immunology, 57:191-280 (1994); Lang et al., J. Biol. Chem., 271:30126-30135 (1996); Rader and Barbas, Phage Display, A Laboratory Manual, Cold Spring Harbor Laboratory Press, Cold Spring Harbor, N.Y. (in press)). Existing pComb vectors are generally not well suited for production of dimeric antibodies, i.e., antibodies having two antigenic binding sites.

There is a continuing need for phagemid vectors which facilitate production of dimeric (divalent) species of biologically active peptides, fusion peptides and combinatorial libraries containing such species.

### SUMMARY

pComb vectors are provided which contain, inter alia, upstream and downstream translatable nucleic acid sequences linked by nucleotides which allow for directional ligation of a desired nucleic acid insert, a dimerization cassette; the downstream insert providing a filamentous phage membrane anchor. Such pComb vectors are useful in situations where dimerized expression products are a desired result.

### BRIEF DESCRIPTION OF THE DRAWINGS

FIG. 1 is a plasmid map illustrating pRL4 (prior art).

FIG. 2 is a full restriction map of a jun dimerization cassette.

FIG. 3 is a schematic diagram showing construction of a PCR fragment containing a flexible linker (murine kappa hinge region) followed by a jun leucine zipper dimerization domain.

FIG. 4 is a plasmid map illustrating pRL4 TT.

FIG. 5 is a schematic flow chart diagram showing construction of pRL8 by insertion of the PCR fragment of FIG. 3 into a pRL4 backbone that had been treated to remove the stuffer region. The resulting construct is cut with restriction endonucleases to remove scFv which is then replaced with a pRL4 stuffer region for light and heavy chain.

FIGS. 6A and 6B depict two Western Blots. FIG. 6A) Reducing gel: lanes 1-5 are test scFv-jun clones from bacterial cell lysate, lane 6 shows the scFv only (no jun domain), lane 7 is from untransformed bacteria TOP10′F , lane 8 is Molecular Weight Markers, lane 9 is control Fab, lanes 10-13 are test scFv-jun clones from bacterial supernates. FIG. 6B) Non-Reducing gel: lane compositions are identical to that in FIG. 6A. HA-tagged antibody fragments were detected and visualized in this blot with anti-HA tag antibody. The result is that clones j-13 and j-18 show dimerization of the scFv on the non-reducing gel as compared to the position of monomeric scFv-jun seen in the reducing gel.

FIG. 7 is a plasmid map illustrating pLR8.

FIG. 8A-C depicts the nucleic acid sequence of pRL8 (SEQ ID NO 8).

FIG. 9 is a schematic diagram showing a portion of pRL8.

FIG. 10 depicts the nucleic acid sequence (SEQ ID NO 9) along with amino acid sequences (SEQ ID NO 10) corresponding to certain delineated nucleic acid sequences of a portion of the region shown in FIG. 9.

FIG. 11 depicts the nucleic acid sequence of the light chain stuffer (SEQ ID NO 11)

FIG. 12 depicts the nucleic acid sequence of the heavy chain stuffer (SEQ ID NO 12).

### DETAILED DESCRIPTION OF PREFERRED EMBODIMENTS

Vectors according to the present invention are especially well suited for production of dimeric polypeptides. In particular, biologically active polypeptides that utilize dimeric motifs for their activity are well suited for production with vectors incorporating a dimerizing domain as described herein. Examples of such biologically active polypeptides include antibodies and fragments thereof. As used herein, “antibodies” or “antibody” refers to entire antibody molecule(s) or molecules that contain immunologically active portions of whole antibody molecules and includes Fab, F(ab′)2, scFv, Fv, heavy chain variable regions and light chain variable regions. The terms “antibody”and “immunoglobulin” are used interchangeably herein. As used herein, “polypeptides” is meant to include short peptides and proteins.

Although the present description is exemplified through pRL8, it is contemplated that any pComb vector can be modified using techniques described herein. These include phagemids based on gene III and gene VIII. Examples of pComb vectors that can be made to incorporate a dimerization domain as described herein include, but are not limited to pComb, pComb8, pComb2-8, pComb3, pComb3H, pComb3X, pComb2-3 and pComb2-3. The pRL8 vector is a modified version of pRL4.

pRL8 contains a dimerization cassette inserted at the Spel site. In an especially preferred embodiment, homo-dimerization of single chain antibodies is accomplished by the addition of the Jun leucine zipper domain, which is responsible for protein-protein interactions. The jun dimerization cassette utilized was described in de Druif, J. and Logtenberg, T. (1996) J. Biol. Chem., 271, pp 7630-7634. FIG. 2 illustrates a full restriction map of the jun dimerization cassette. Dimerization cassettes according to the present invention can also include other dimerization domains, i.e., there are a number of dimerization domains (lexA, Zn fingers, fos, jun etc.) that can be utilized in these vectors to obtain multivalency of antibody fragments. Examples of dimerization domains include, but are not limited to, the following: jun (DeKruif, J. and Logtenberg, T. J. Biol. Chem. 271:7630-7634, 1996; Kostelny, S. A., Cole, M. S., and Tso, J. Y. J.Immunol -148:1547-1553, 1992) the LexA dimerization region (Kim, B. and Little, J. W. Science 255:203-206, 1992), the yeast GCN4 dimerization domain (van Heeckeren, W. J. Sellers, J. W., Struhl, K. Nucleic Acids Res. 20:3721-3724, 1992), Gin invertase from the bacteriophage Mu (Spaeny-Dekking, L., Schlicher, E., Franken, K., van de Putte, P., Goosen, N. J. Bacteriol. 34:1779-1786, 1995), E. coli NTRC protein dimerization domain (Klose, K. E., North, A. K., Stedman, K. M., Kustu, S. J. Mol. Biol. 241:233-245,1994), and HSV-1 ICP4 dimerization domain (Gallinari, P., Wiebauer, K., Nardi, M. C., Jiricny, J. J. ViroL 68:3809-3820, 1994) all incorporated by reference. Also, a high temperature dimer domain from thermus organisms can be utilized (MacBeath, G., Kast, P., Hilvert, D., Biochemistry 37:100062-73, 1998 and MacBeath, G., Kast, P., Hilvert, D., Science 279:1958-61, 1998). These are functional domains that, when incorporated into a molecule, allow for dimerization to occur. Those of ordinary skill in the art are familiar with these and other dimerization domains.

In constructing pRL8, both the dimerization cassette and a single chain antibody were PCR generated using the protocol detailed below. PCR was performed to generate several initial product fragments, as illustrated in FIG. 3. Using pRL4-TT Fab (anti-tetanus toxoid) as a template (see FIG. 4), reactions were set up to generate fragment #1 using primers OmpA Seq F (ACA GCT ATC GCG ATT GCA GTG GCA C) (SEQ ID NO 1) and HKJo-BL (GGA AGA TCT AGA GGA ACC ACC CCC ACC ACC GCC CGA GCC ACC GCC ACC AGA GGA AGT TCG TTT GAG TTC CAC CTT G) (SEQ ID NO 2); fragment #2 using primers HSCVH (GGT GGT TCC TCT AGA TCT TCC GAG GTG CAG CTG CTC GAG) (SEQ ID NO 3) and Jun 1 Rev (CCT CCA GAC GGG CGA TGC GGC CAC CGC AGG MG AGC CCG GTG GGG TAG ACG GTT TCG GAC TAG TGG AGA CGG TGA CGG TGG TCC) (SEQ ID NO 4); and fragment #3 with Jun3 For (ACT GCC MC ATG CTG CGC GM CAG GTG GCA CAG CTG AAA CAG AM GTT ATG MC CAT GGC GGT TGT GCT AGT GGC CAG GCC GGC CAG) (SEQ ID NO 5) and NPC amb-B (GCT TAC MT TTC CCA GAT CTG CG) (SEQ ID NO 6). The fusion PCR to generate fragment #4 was then performed on PCR product fragment #1 and #2 with primers OmpA Seq F and Jun 2 Rev (TCG CGC AGC ATG TTG GCA GTG GAC GCC AGC TCG GAG TTC TGA GCT TTC AGG GTT TTC ACT TTT TCC TCO AGA CGG GCG ATG CGG) (SEQ ID N07). The full fragment #5 was created by mixing fragment #4 and fragment #3 together with primers OmpA SeqF and NPC3amb-B.

As is shown in the schematic flowchart of FIG. 5, the final PCR product was digested with Sfi I and ligated into pRL4 backbone that had been Sfi I treated to remove the stuffer region. Following ligation, the DNA was electroporated into Top10F′ bacteria (Invitrogen, Carlsbad, Calif.) and plated for single colonies. 40 colonies were picked the next day and grown in media overnight. The next morning, the bacterial supernates were harvested and used in an ELISA to identify those bacterial clones producing scFv able to bind to the immobilized antigen. Several clones were further analyzed by Western Blot for detection of dimerized scFv with an anti-HA tag antibody (HA, 11, Babco, Berkeley, Calif.). Results of the Western blot are depicted in FIG. 6. Two clones were sequence analyzed. Clone 13 was found to contain the correct dimerization domain sequence.

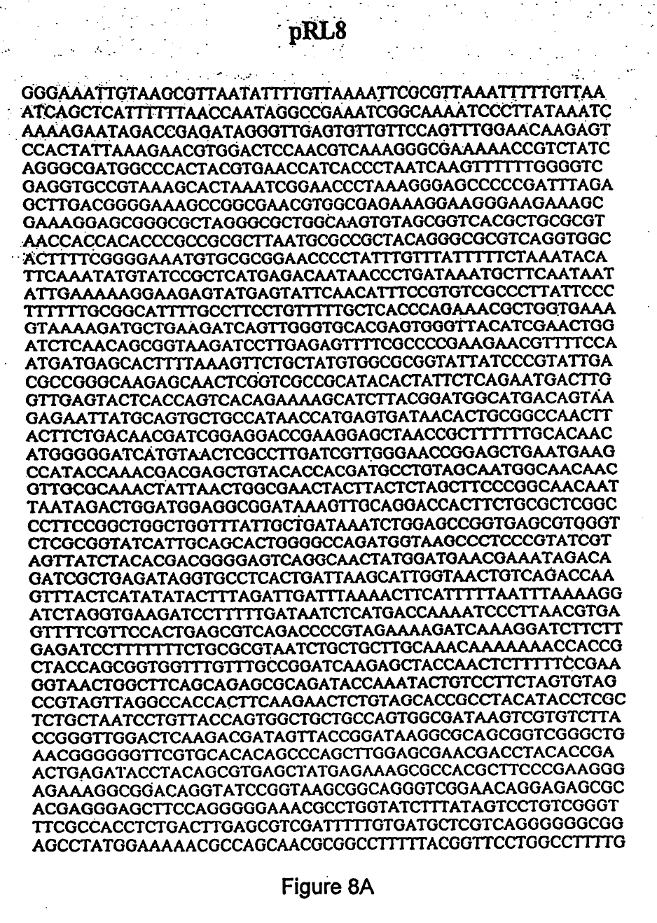

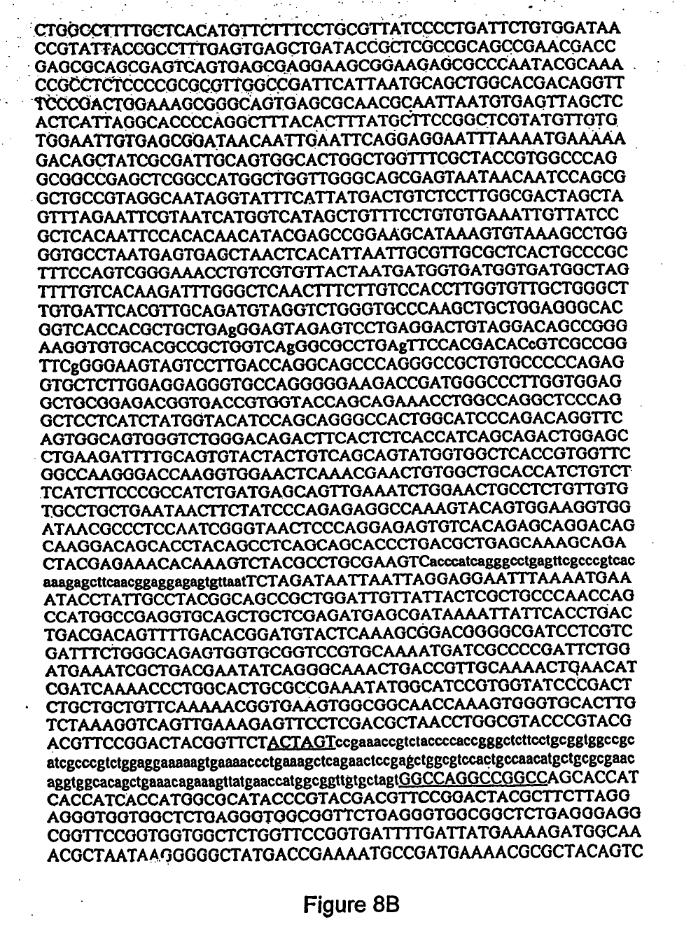

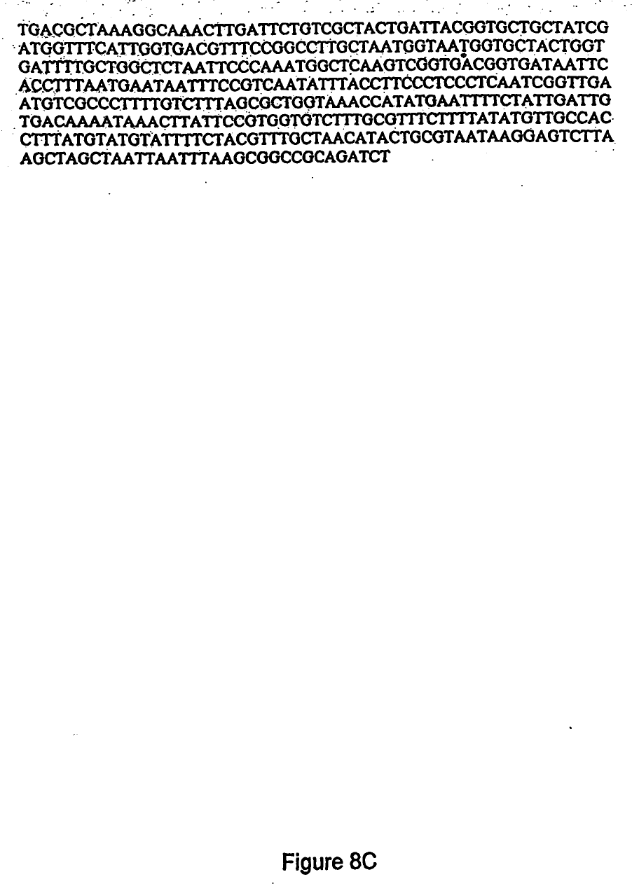

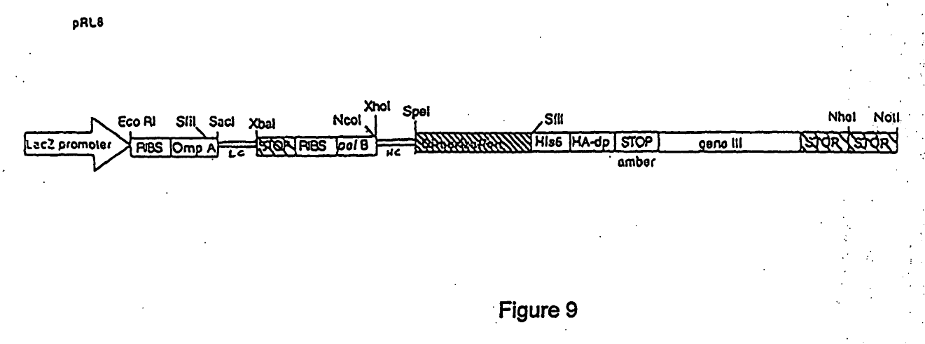

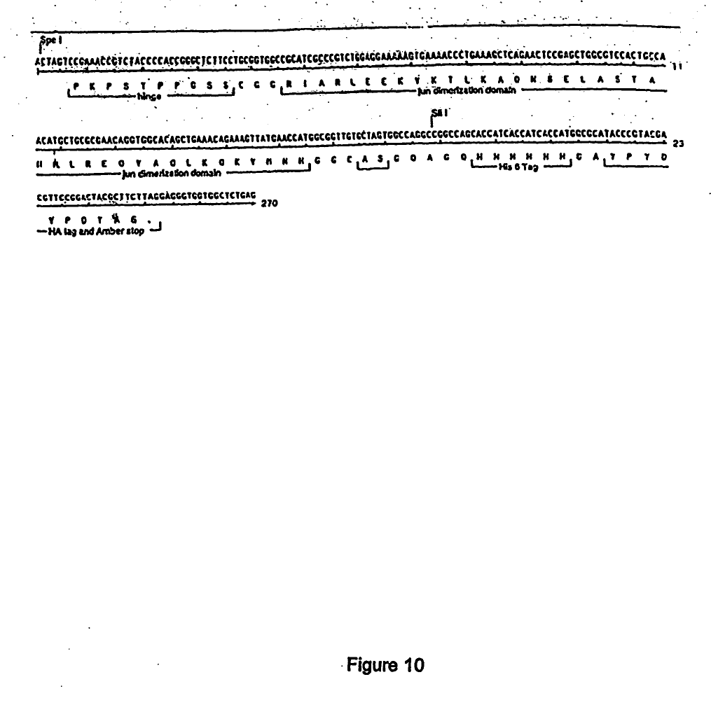

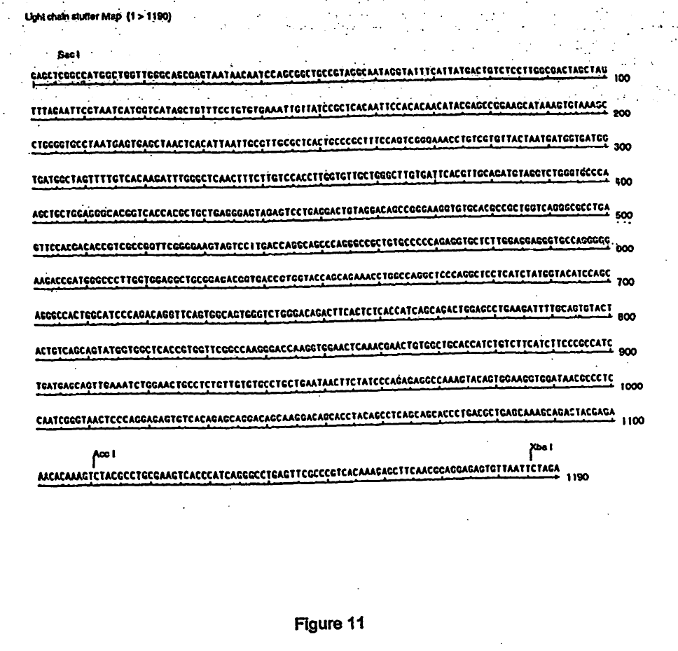

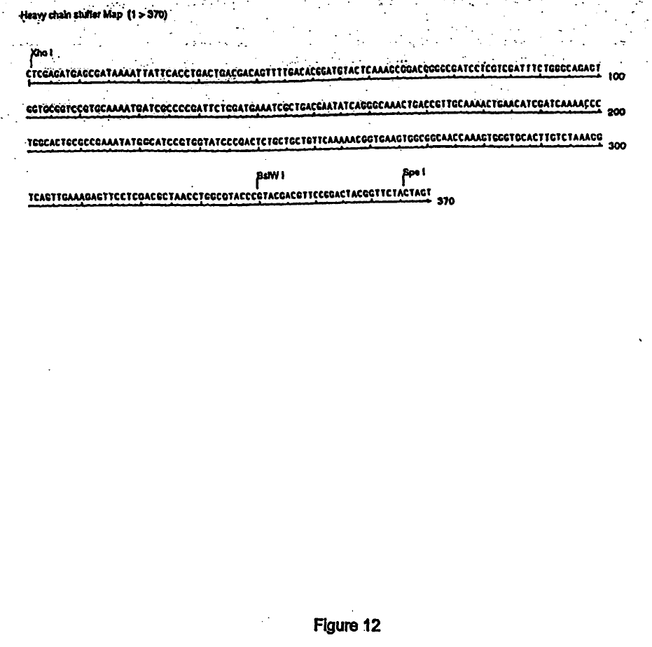

PRL8 was then made for general use by removing the scFv with a Sac I and Spe I digest. In place of the antibody, a stuffer region was inserted into pRL8. The stuffer used was that contained in the starting plasmid pRL4 in the Sac I to Spe I region. FIG. 11 depicts the nucleic acid sequence of the light chain stuffer (SEQ ID NO 11). FIG. 12 depicts the nucleic acid sequence of the heavy chain stuffer (SEQ ID NO 12). FIGS. 7 through 10 provide maps and sequence information for pRL8. The final pRL8 vector differs from pRL4 only in the region between the Spe I and downstream Sfi I site.

Phagemids having a dimerization domain according to the present invention are especially useful in the production of biologically active molecules such as antibody fragments which may require dimerization in order to crosslink for activation of target receptors. Thus, the present invention provides an efficient modality for production of multivalent antibody fragments. It is contemplated that phagemids according to the present invention may be used in connection with production and screening of libraries made in accordance with phage display technology. See, e.g., Cwirla et al., Proc. Natl. Acad. Sci. USA 87, 6378-6382 (1990); Barbas et al., Proc. Natl. Acad. Sci. USA, 88, 7978-7982 (1991). See also, Pluckthun, Biochemistry, 31:1579-1584 (1992); Holland et al., Current Opin. Biotech., 4:446-449 (1993). In one embodiment, following the panning or sorting steps of ScFv or Fab libraries, the library of panned molecules are suitably restricted with restriction endonucleases and cloned into pComb vectors incorporating one or more dimerization domains according to the present invention. Transformation of a suitable prokaryotic host allows expression of dimeric soluble binding antibody fragments for analysis in bioassays. The antibody fragments are then transported to the periplasmic space and form dimers there. In the case of pRL8, the library of panned molecules are restricted with Sac I and Spe I and cloned into pRL8. Subcloning to pRL8 individually or en masse following FACS sorting or panning allows expression, e.g., of dimeric soluble Fabs.

It will be understood that various modifications may be made to the embodiments described herein. Therefore, the above description should not be construed as limiting, but merely as exemplifications of preferred embodiments. Those skilled in the art will envision other modifications within the scope and spirit of the claims appended hereto.

1. A pComb family phagemid comprising nucleic acid encoding a dimerization domain.

2. A pComb family phagemid according to claim 1 wherein the dimerization domain is selected from the group consisting of LexA, GCN4 dimerization domain, Mu gin invertase, E coli NTRC protein dimerization domain, HSV-1 lcp4 dimerization domain, CH3 dimerization domain, Zn fingers and fos.

3. A pComb family phagemid according to claim 1 wherein the dimerization domain is a jun leucine zipper domain.

4. A pComb family phagemid according to claim 1 further comprising nucleic acid encoding at least a portion of an antibody.

5. A pComb family phagemid according to claim 1 wherein the phagemid is pRL8.

6. A pComb family phagemid according to claim 1 wherein the phagemid is selected from the group consisting of pComb, pComb8, pComb2-8, pComb3, pComb3X, pComb3H, pComb2-3 and pComb2-3′.

7. A host cell transformed with a phagemid according to claim 1.

8. A dimeric polypeptide produced by a host cell according to claim 7.

9. A phagemid display library manufactured using a phagemid according to claim 1.
